(19) United States
(12) Patent Application Publication
(10) Pub. No.: US 2024/0065827 A1
(43) Pub. Date: Feb. 29, 2024

(54) Accommodating Intraocular Lens Comprising a Combination of Multiple Variable Lenses

(71) Applicant: AKKOLENS INTERNATIONAL B.V., Breda (NL)

(72) Inventors: Michiel Christiaan Rombach, Breda (NL); Willem Pieter Van Lawick, Breda (NL)

(21) Appl. No.: 18/237,925

(22) Filed: Aug. 25, 2023

(30) Foreign Application Priority Data
Aug. 26, 2022 (NL) 2032859

(51) Int. Cl. A61F 2/16

(52) U.S. Cl. CPC A61F 2/1629; A61F 2/1651; A61F 2002/1682; A61F 2210/0057; A61F 2230/0071; A61F 2250/0018

(57) ABSTRACT

Accommodating intraocular lens including a combination of variable lenses. Firstly, a variable lens including two spherical lenses fitted onto two optical elements to provide variable optical power of which the degree depends on the degree of movement of at least one of spherical lens along the optical axis. Secondly, a variable lens comprising at least two cubic surfaces fitted onto the same two optical elements which provides a lens of variable optical power of which the degree depends on the degree of movement of at least one of the optical elements in a direction perpendicular to the optical axis. The combination of variable lenses is fitted into a mechanical construction providing said movements of the optical elements.

### CROSS-REFERENCE TO RELATED APPLICATION

This application claims priority to the Netherlands Patent Application No. 2032859 filed Aug. 26, 2022, the disclosure of which is hereby incorporated by reference in its entirety.

### BACKGROUND OF THE INVENTION

### Field of the Invention

The present invention relates to an accommodating intraocular lens comprising a combination of variable lenses (optical elements) including, firstly, a variable lens comprising two spherical lenses fitted onto two optical elements to provide variable optical power of which the degree depends on the degree of movement of at least one of spherical lens along the optical axis and, secondly, a variable lens comprising at least two cubic surfaces fitted onto the same two optical elements which provides a lens of variable optical power of which the degree depends on the degree of movement of at least one of the optical elements in a direction perpendicular to the optical axis. The combination of variable lenses is fitted into a mechanical construction providing said movements of the optical elements.

### Description of Related Art

Accommodating intraocular lenses provide the eye with adjustment of focus power. Such lenses can comprise multiple optical elements each fitted with at least one cubic optical shape or partially cubic function shaped or free-formed. The combination of, for example, two, such cubic optical shapes provide a lens of variable optical power with the degree of optical power depends on the degree of movement, in this example shift, of the optical elements in, opposite, lateral directions, meaning: directions perpendicular to the optical axis, henceforth abbreviated as the ‘lateral variable lens’. Such variable lenses are disclosed in, for example, US2008046076 and WO2005084587 and related documents.

Such lenses can, alternatively, also comprise multiple optical elements each fitted with at least one largely spherical optical shape. The combination of, for example, two, such spherical optical shapes provide a lens of variable optical power with the degree of optical power depending on the degree of movement, in this example axial travel, of the optical elements in, generally opposite but not necessarily so, axial direction, meaning: directions along the optical axis, henceforth abbreviated as the ‘axial variable lens’. Such variable lenses are disclosed in, for example, WO2011062486 and US20020002404 and related documents.

### SUMMARY OF THE INVENTION

It is a first object of the present invention to provide a lens construction that can provide improved variable optical power.

The present invention provides thereto an accommodating intraocular lens construction comprising an optical axis with the lens comprising at least two optical elements with each element comprising two optical surfaces,
wherein the optical elements are mutually elastically coupled by at least one elastic haptic, and
wherein at least one, preferably each, optical element comprises at least two optical subelements wherein at least one optical subelement comprises an optical surface that is at least partially cubic function shaped or free-formed and wherein at least one other optical subelement comprises at least one optical surface that is at least partially spherical shaped, and
wherein the elastic haptic is configured to provide, at compression of the lens construction in a lateral direction, said lateral direction being directed perpendicular to the optical axis, a combination of lateral movements in lateral direction and axial movements in axial direction of the optical elements of the optical elements,
wherein the axial movements are directed along the optical axis.

The present invention discloses an intraocular lens construction. The lens construction is advantageous because it can provide variable optical power due to the combination of at least one lateral variable lens (optical element) and at least one axial variable lens (optical element) and an elastic haptic. The lens construction according to the present invention provides said, independent, movements of the optical elements. The lens construction according to the present invention can provide a combination of lateral and axial movements of the optical elements and as such provide a variable optical power.

Each optical element according to the present invention comprises at least two, preferably two, optical surfaces. One of these surfaces is at least partially spherical shaped. Preferably, this surface is the outer surface, facing outwards the lens construction. Another optical surface of the same optical element is at least partially formed according to the shape of a graph resulting from a cubic formula, which can be referred to as cubic optical shape in this document and/or as partially cubic function shaped and/or free-formed. The partially cubic function shaped or free-formed is preferably positioned as the inner surface in the optical element, facing inwards the lens construction. The at least partially cubic function shaped or free-formed surface is preferably part of an optical subelement that has at least partially the shape of an area under a graph of a cubic formula.

The at least partially spherical shaped surface can form part of another optical subelement that has at least partially the shape of an area under a graph of a spherical formula.

In the present document and illustrations embodiments of the present invention are described. In a preferred embodiment of the present invention the lens construction comprises two independent hinges to allow for the two independent directions of movement of the optical elements.

In an embodiment, the present invention relates to an accommodating intraocular lens construction having an optical axis with the lens comprising at least two optical elements with each element comprising two optical surfaces with the optical elements mutually elastically coupled by at least one elastic haptic with each optical element comprising two optical surfaces with element fitted with at least one at least partially (largely) partially cubic function shaped or free-formed and at least one at least partially (largely) spherical optical shape wherein the elastic haptic is adapted to provide, at compression of the lens construction in a lateral direction, directed perpendicular to the optical axis, a combination of lateral movements of the optical elements and axial movements of the optical elements in a axial direction, directed along the optical axis with the lens construction.

The lens construction according to the present invention may comprise two haptics to fit the lens construction according to the present invention at the sulcus plane of the eye.

Typically, the lens construction according to the present invention comprises a combination of at least two at least partially (largely) cubic function shaped or free-formed surfaces, wherein preferably said surfaces are configured such that a lens of variable optical power is provided of which the degree of optical power depends on the degree of mutually opposite movements of the optical elements in a lateral direction.

Optionally, the lens construction may comprise a combination of at least two at least partially (largely) spherical optically shaped surfaces that are configured to provide a lens of variable optical power of which the degree of optical power can depend on the degree of movements of the elements in an axial direction.

In an embodiment, the spherically optically shaped surfaces may be configured such that a lens of variable power is provided of which the degree of optical power depends on the degree of movements of the elements in axial direction, wherein the axial movements of the optical elements can be movements in opposite directions along the optical axis or alternatively, wherein the axial movements of the optical elements are in the same direction along the optical axis.

In another embodiment, the lens construction may comprise a combination of at least two substantially spherical optically shaped surfaces is configured to provide a lens of variable optical power of which the degree of optical power depends on the degree of movements of the elements in an axial direction wherein the axial movements of the optical elements are in the same direction along the optical axis.

In another embodiment, both optical elements can comprise a spherical optical shape of a positive optical power. Preferably, a the lens construction according to the present invention is configured to move both elements in the same direction along the optical axis. Optionally at least one other optical element comprises a spherical optical shape of a negative optical power and preferably the lens construction can be (mechanically) configured to move both elements in opposite directions along the optical axis.

In an embodiment, at least two additional free-form optical surfaces can also be fitted onto the two at least partially (largely) partially cubic function shaped or free-formed surfaces.

These free-form optical surfaces can provide variable optical power of at least one additional aberration of which the degree of optical power depends on the degree of mutually opposite movements of the elements in a lateral direction. The lateral direction meant is a direction perpendicular to the optical axis. For example the additional free-form surfaces can provide variable correction of, undesired, variable astigmatism and/or coma due to lateral shift of spherical lenses included in the lens construction, or example, lateral shift versus each other and/or lateral shift versus the cornea of the eye.

In an embodiment of the lens construction, one optical element can comprise a spherical optical shape of a positive optical power with at least one other element comprising a spherical optical shape of a negative optical power with mechanical construction configured to move at least the two (both) elements in opposite directions along the optical axis with the optical elements fitted into the (mechanical) lens construction adapted to provide a combination of lateral and axial movements of the optical elements. Preferably, in a preferred embodiment, the lens construction may comprise a combination of at least two, preferably two, sets of hinges comprising at least one (set of) hinge(s) adapted to provide lateral movement of the optical elements and at least one (set of) hinge(s) adapted to provide axial movement of the optical elements. Preferably an embodiment of the (mechanical) lens construction, if hinges are applied, comprise two independent sets of hinges.

In an embodiment of the lens construction according to the present invention, at least one optical surface is shaped like a cubic chape function. Preferably at least one optical surface may be shaped as a Hermitian shape function. At least one optical surface may be shaped as the area under the graph of a Hermitian shape function.

### BRIEF DESCRIPTION OF THE DRAWINGS

Further advantages, features and details of the invention are elucidated on the basis of preferred embodiments thereof, wherein reference is made to the accompanying drawings, in which:

FIG. 1 schematically shows a perspective view of a lens construction according to the present invention;

FIG. 2 schematically shows a cross section of a lens construction and the effect thereof according to the present invention.

### DESCRIPTION OF THE INVENTION

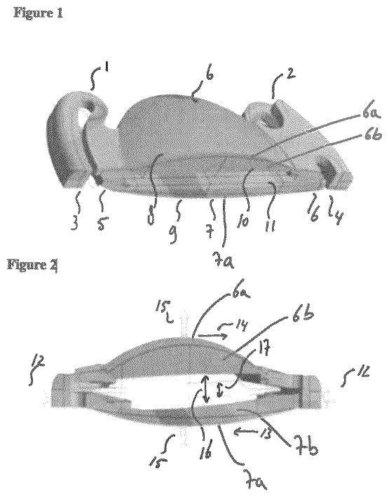

FIG. 1 shows an example of an accommodating intraocular lens construction as disclosed in the present document with the lens comprising, in this example, a (mechanical) construction including two haptics, 1,2, to fit the lens at the sulcus plane of the eye, the plane in front of the capsular bag and behind the iris of the eye. The (mechanical) lens construction shown includes a first set of elastical hinges, 3,4, to provide lateral movement of the optical elements and a set of hinges, 5,6, to provide axial movement of the optical elements. The optical elements, 6,7, comprise two spherical optical surfaces, 8,9, and two cubic shaped optical surfaces, 10, 11, forming the intra-lenticular space 16. The two optical surfaces 10,11 shown are cubic function or free-from shaped and have as such the shape of the area under the graph a cubic function. In the presentation shown, the upper optical element 6 comprises one subelement 6a comprising a spherical optical surface 8 and the same optical element comprises another subelement 6b comprising a cubic shaped surface 10. In the presentation shown, the lower optical element 7 comprises one subelement 7a comprising a spherical optical surface 9 and the same optical element comprises another subelement 7b comprising a cubic shaped surface 11.

FIG. 2 shows the effect of lateral compression of the lens construction, 12, deforming the lens construction. The optical elements shift in lateral and opposite directions, 13, 14, and both the optical elements move in the axial direction, 15, which mutual displacement increases the intra-lenticular space 16,17 between the optical elements 6,7. The size of the space after deforming is shown with reference number 16, which is an increased size with respect to the also shown size of the space 17 that shows the size of the intra-lenticular space in case the lens construction is at rest.

1. An accommodating intraocular lens construction comprising an optical axis with the lens comprising at least two optical elements with each element comprising two optical surfaces,
wherein the optical elements are mutually elastically coupled by at least one elastic haptic, and
wherein at least one, preferably each, optical element comprises at least two optical subelements wherein at least one optical subelement comprises an optical surface that is at least partially cubic function shaped or free-formed and wherein at least one other optical subelement comprises at least one optical surface that is at least partially spherical shaped, and
wherein the elastic haptic is configured to provide, at compression of the lens construction in a lateral direction, said lateral direction being directed perpendicular to the optical axis, a combination of lateral movements in lateral direction and axial movements in axial direction of the optical elements of the optical elements,
wherein the axial movements are directed along the optical axis.

2. The lens construction according to claim 1, comprising two optical elements, wherein the two optical elements are positioned on opposite sides of the lens construction and are divided by an intra-lenticular space between the optical elements.

3. The lens construction according to claim 2, wherein the optical elements are configured to shift in lateral and opposite directions such that and both the optical elements move in the axial direction which mutual displacement increases the intra-lenticular space between the optical elements.

4. The lens construction according to claim 1 wherein a combination of at least two at least partially cubic function shaped or free-formed shaped surfaces is configured to provide a lens of variable optical power of which the degree of optical power depends on the degree of mutually opposite movements of the optical elements in a lateral direction.

5. The lens construction according to claim 1 wherein a combination of at least two at least partially spherical optically shaped surfaces is configured to provide a lens of variable optical power of which the degree of optical power depends on the degree of movements of the elements in an axial direction.

6. The lens construction according to claim 4 wherein the axial movements of the optical elements are movements in opposite directions along the optical axis.

7. The lens construction according to claim 4 wherein the axial movements of the optical elements are in the same direction along the optical axis.

8. The lens construction according to claim 1 wherein the at least two optical elements comprise a spherical optical shape of a positive optical power and wherein the lens construction provided with a mechanical construction configured to move at least two optical elements in the same direction along the optical axis.

9. The lens construction according to claim 1 wherein at least one optical element comprises a spherical optical shape of a positive optical power and wherein at least one other optical element comprises a spherical optical shape of a negative optical power, wherein the lens construction is adapted to move both elements in opposite directions along the optical axis.

10. The lens construction according to claim 1 wherein the optical elements also comprise at least two additional free-form optical surfaces configured to provide a variable optical power of at least one additional aberration of which the degree of optical power depends on the degree of mutually opposite movements of the elements in a lateral direction.

11. The lens construction according to claim 9 wherein the additional free-form surfaces are configured to provide variable correction of variable astigmatism.

12. The lens construction according to claim 9 wherein the additional free-form surfaces are configured to provide variable correction of variable coma.

13. The lens construction according to claim 1, wherein the optical subelements within one optical element are in abutting contact (in the lens construction) and are configured for providing a combination of lateral and axial movements of the optical elements and/or of the optical subelements.

14. The lens construction according to claim 1 wherein the lens construction comprises a combination of at least two independent sets of hinges, in particular two independent sets of hinges.

15. The lens construction according to claim 13, wherein the at least two independent sets of hinges comprise one set of hinges adapted to provide lateral movement of the optical elements and one set of hinges adapted to provide axial movement of the optical elements.

16. The lens construction according to claim 1, wherein at least one optical surface is shaped like a cubic chape function, in particular like a Hermitian shape function.
